(19) United States
(12) Patent Application Publication
(10) Pub. No.: US 2022/0396784 A1
(43) Pub. Date: Dec. 15, 2022

(54) DEVICE, SYSTEM AND METHOD FOR ISOLATING A BIOLOGICAL MATERIAL

(71) Applicant: One Biomed PTE LTD, Singapore (SG)

(72) Inventors: Benjamin A. Reisman, Carlsbad, CA (US); Espir Kahatt, Carlsbad, CA (US); Mi Kyoung Park, Singapore (SG)

(21) Appl. No.: 17/777,230

(22) PCT Filed: Feb. 22, 2021
(86) PCT No.: PCT/SG2021/050085

§ 371 Date: May 16, 2022

Related U.S. Application Data
(60) Provisional application No. 62/982,259, filed on Feb. 27, 2020.

(51) Int. Cl. C12N 15/10; B01L 3/00

(52) U.S. Cl. CPC C12N 15/1006; B01L 3/50273; B01L 2200/16; B01L 2300/042; B01L 2300/0681; B01L 2300/0883; B01L 2400/0683

(57) ABSTRACT

A device for isolating a biological material from a sample includes a housing, a slider and a dry reagent capsule. The housing defines a plurality of compartments and a plurality of fluid channels. Each compartment is configured to be fluidically connected to a respective fluid channel, and each fluid channel includes a respective end terminating at a track disposed on the housing. The slider is movable along the track and includes a plurality of connecting channels extending therethrough. A selected one of the connecting channels is configured to connect ends of selected ones of the fluid channels based on a position of the slider along the track. The dry reagent capsule is configured to be mounted to the housing, and includes at least one dry reagent for mixing with the sample. The dry reagent capsule is further configured to be fluidically connected to a respective fluid channel in-situ.

### CROSS REFERENCE

This application is a national stage entry application under 35 U.S.C. 371 of PCT Patent Application No. PCT/SG2021/050085, filed 22 Feb. 2021, which claims priority to U.S. Provisional Patent Application No. 62/982,259, filed 27 Feb. 2020, the entire contents of each of which are incorporated herein by reference.

### TECHNICAL FIELD

The present disclosure relates broadly, but not exclusively, to devices, systems and methods for isolating a biological material, such as a nucleic acid.

### BACKGROUND

Nucleic acid isolation is the first step that needs to be performed for many modern genomics techniques and applications. After lysis of cells, deoxyribonucleic acid (DNA) and ribonucleic add (RNA) need to be isolated and purified for subsequent downstream processing and analysis, such as PCR and sequencing.

Automated nucleic acid extraction systems have the potential to improve workflow and decrease variability in the basic research as well as in the clinical laboratory. While many automated systems are commercially available, most of the systems are based on automatic liquid handling technology that involves pipetting and dispensing of samples and bio-reagents, which can be prone to cross-contamination and require vigorous maintenance such as pre- and post-clean-up by end-users.

A need therefore exists to provide devices, systems and methods that can address at least some of the above problems.

### SUMMARY

An aspect of the present disclosure provides a device for isolating a biological material from a sample. The device comprises a housing defining a plurality of compartments and a plurality of fluid channels, wherein each compartment is configured to be fluidically connected to a respective fluid channel, and wherein each fluid channel comprises a respective end terminating at a track disposed on the housing; a slider movable along the track, the slider comprising a plurality of connecting channels extending therethrough, wherein a selected one of the connecting channels is configured to connect ends of selected ones of the fluid channels based on a position of the slider along the track; and a dry reagent capsule configured to be mounted to the housing, the dry reagent capsule comprising at least one dry reagent for mixing with the sample, wherein the dry reagent capsule is further configured to be fluidically connected to a respective fluid channel in-situ.

The housing may comprise a first housing member securely joined to a second housing member, and the first housing member may comprise grooves arranged to form the respective fluid channels.

The plurality of compartments may comprise a sample compartment configured to receive the sample, a plurality of liquid reagent compartments and a waste compartment.

Each of the sample and liquid reagent compartments may comprise a respective inlet configured to be connected to pneumatic source for controlling a fluid flow to or from said compartment.

The device may further comprise a first pneumatic vent disposed in one of the liquid reagent compartments and a second pneumatic vent disposed in the waste compartment.

The plurality of liquid reagent compartments may be preloaded with respective liquid reagents.

In a first position, the slider may be configured to connect a first liquid reagent compartment containing a hydration buffer with a first chamber of the dry reagent capsule, the first chamber containing a first dry reagent, for mixing the hydration buffer with the first dry reagent to form a first solution.

In a second position, the slider may be configured to connect the first liquid reagent compartment with a second liquid reagent compartment containing a lysis buffer, for mixing the first solution with the lysis buffer to form a second solution.

In a third position, the slider may be configured to:

connect the second liquid reagent compartment with a second chamber of the dry reagent capsule, the second chamber containing a second dry reagent, for mixing the second solution with the second dry reagent to form a third solution; and

connect the second chamber of the dry reagent capsule with the sample compartment for mixing the third solution with the sample to form a fourth solution.

The fluid channels may comprise a binding channel, and in a fourth position, the slider may be configured to connect the sample compartment with the binding channel to store the fourth solution in the binding channel for a predetermined period for extracting the biological material from the fourth solution and binding the extracted biological material to a surface of the binding channel.

In a fifth position, the slider may be configured to:

connect a third liquid reagent compartment containing a first wash buffer with the binding channel to wash the biological material bound to the surface of the binding channel; and

connect the binding channel with the waste compartment for discarding a first waste solution without the biological material to the waste compartment.

In a sixth position, the slider may be configured to:

connect a fourth liquid reagent compartment containing a second wash buffer with the binding channel to wash the biological material bound to the surface of the binding channel; and

connect the binding channel with the waste compartment for discarding a second waste solution without the biological material to the waste compartment.

In a seventh position, the slider may be configured to:

connect a fifth liquid reagent compartment containing an elution buffer with the binding channel to elute the biological material from the surface of the binding channel; and

connect the binding channel with an outlet for collecting the eluted biological material.

In an alternate first position, the slider may be configured to:

connect a first liquid reagent compartment containing a lysis buffer with a chamber of the dry reagent capsule, the chamber containing the at least one dry reagent, for mixing the lysis buffer with the at least one dry reagent to form a reagent solution; and

connect the chamber of the dry reagent capsule with the sample compartment for mixing the reagent solution with the sample to form a sample solution.

The fluid channels may comprise a binding channel, and in an alternate second position, the slider may be configured to connect the sample compartment with the binding channel to store the sample solution in the binding channel for a predetermined period for extracting the biological material from the sample solution and binding the extracted biological material to a surface of the binding channel.

In an alternate third position, the slider may be configured to:

connect a second liquid reagent compartment containing a first wash buffer with the binding channel to wash the biological material bound to the surface of the binding channel; and

connect the binding channel with the waste compartment for discarding a first waste solution without the biological material to the waste compartment.

In an alternate fourth position, the slider may be configured to:

connect a third liquid reagent compartment containing a second wash buffer with the binding channel to wash the biological material bound to the surface of the binding channel; and

connect the binding channel with the waste compartment for discarding a second waste solution without the biological material to the waste compartment.

In an alternate fifth position, the slider may be configured to:

connect a fourth liquid reagent compartment containing a third wash buffer with the binding channel to wash the biological material bound to the surface of the binding channel; and

connect the binding channel with the waste compartment for discarding a third waste solution without the biological material to the waste compartment.

In an alternate sixth position, the slider may be configured to:

connect a fifth liquid reagent compartment containing an elution buffer with the binding channel to elute the biological material from the surface of the binding channel; and

connect the binding channel with an outlet for collecting the eluted biological material.

The at least one dry reagent may comprise lyophilized beads containing a crosslinker selected to attach to the biological material, and the surface of the binding channel may be coated with a functional group selected to attach to the crosslinker.

The biological material may comprise a nucleic acid.

Another aspect of the disclosure provides an automated biological material extraction system comprising:

a receptacle configured to receive the device as described above;

a pressure source configured to control a fluid flow to and from a selected compartment of the sample compartment and liquid reagent compartments; and

an actuator configured to move the slider of the device to predetermined positions along the track.

The system may further comprise a mechanism configured to exert a force on the dry reagent capsule to break a seal covering at least one chamber of the capsule to fluidically connect the capsule with the respective fluid channel in-situ.

The biological material may comprise a nucleic acid.

Another aspect of the present disclosure provides a method of isolating a biological material from a sample, the method comprising:

disposing the sample in the sample compartment of the device as described above;

breaking a seal covering at least one chamber of the dry reagent capsule to fluidically connect the capsule with the respective fluid channel in-situ; and

moving the slider to predetermined positions along the track to:
mix the liquid reagents and the at least one dry reagent with the sample for extracting the biological material from the sample;
bind the extracted biological material to a surface of a binding channel disposed in the device;
purify the biological material bound to the surface of the binding channel; and
elute the purified biological material.

The biological material may comprise a nucleic acid.

### BRIEF DESCRIPTION OF THE DRAWINGS

Embodiments will be better understood and readily apparent to one of ordinary skill in the art from the following written description, by way of example only, and in conjunction with the drawings, in which:

FIG. 1A shows a top perspective view of a device for isolating a biological material according to an example embodiment.

FIG. 1B shows a bottom perspective view of the device of FIG. 1A.

FIG. 10 shows an exploded top view of the device of FIG. 1A.

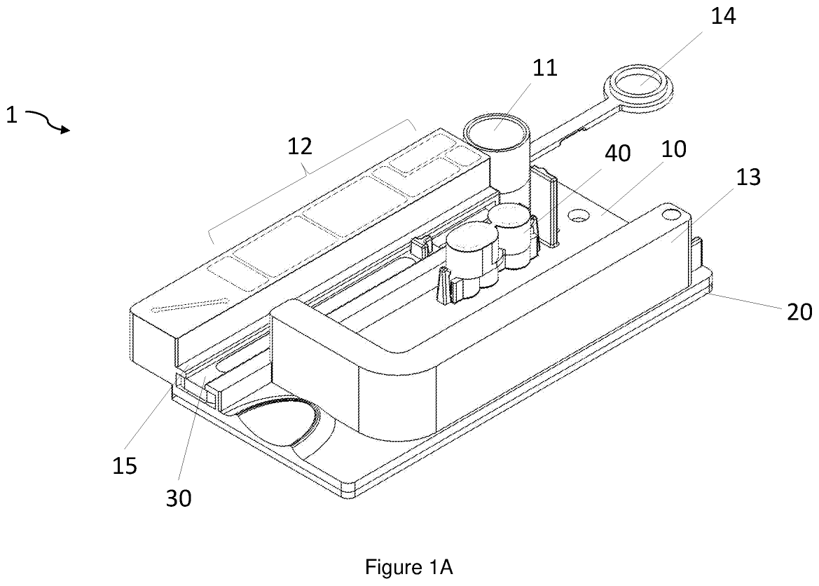

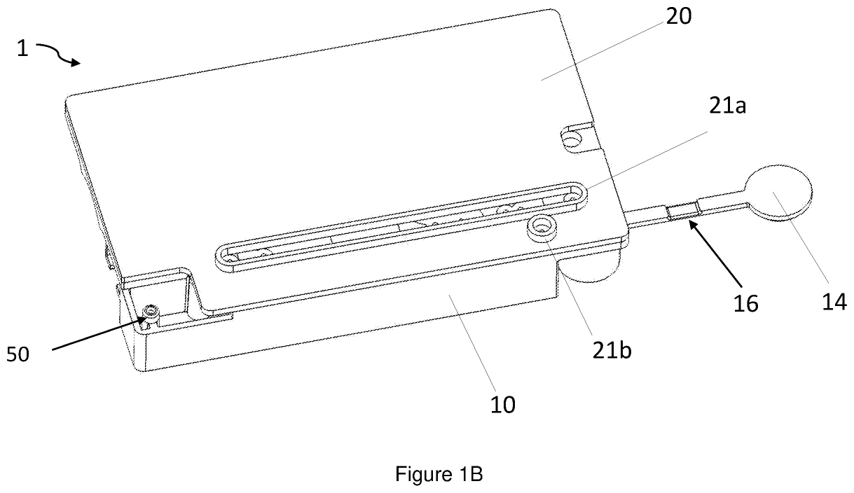

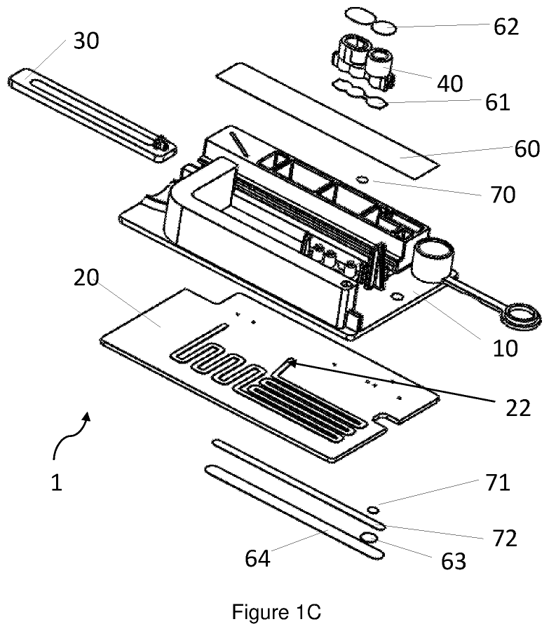

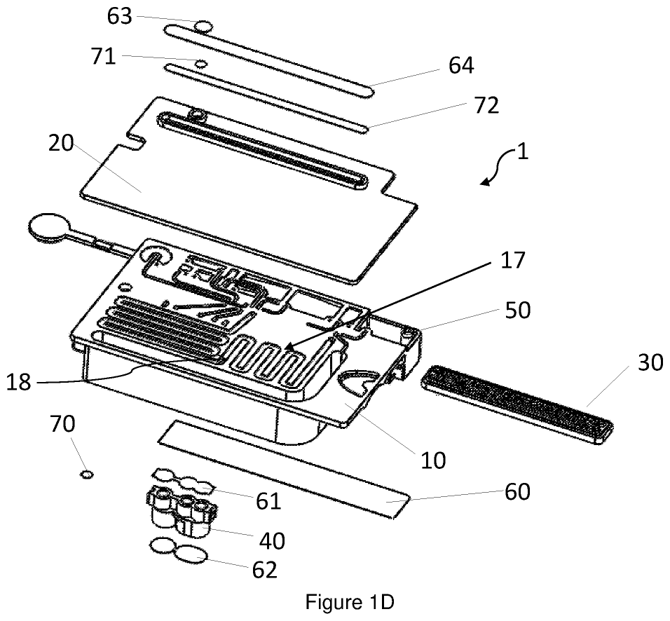

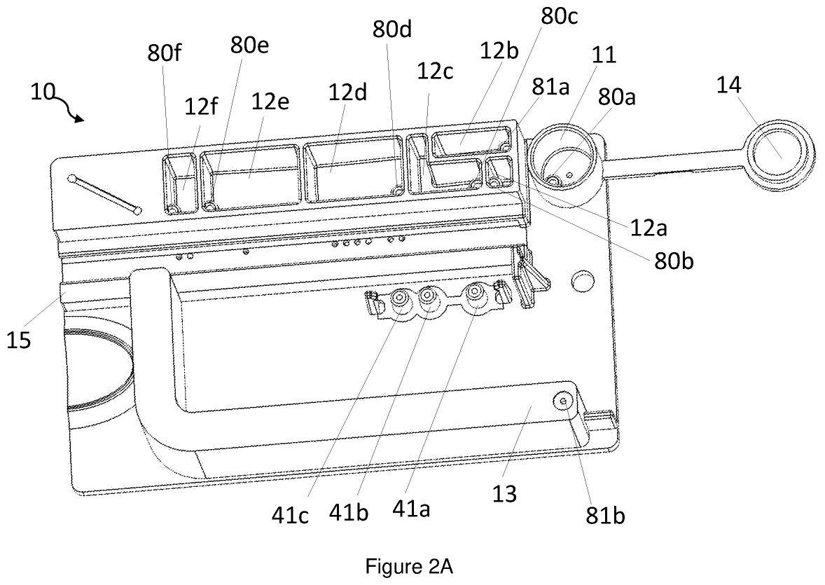

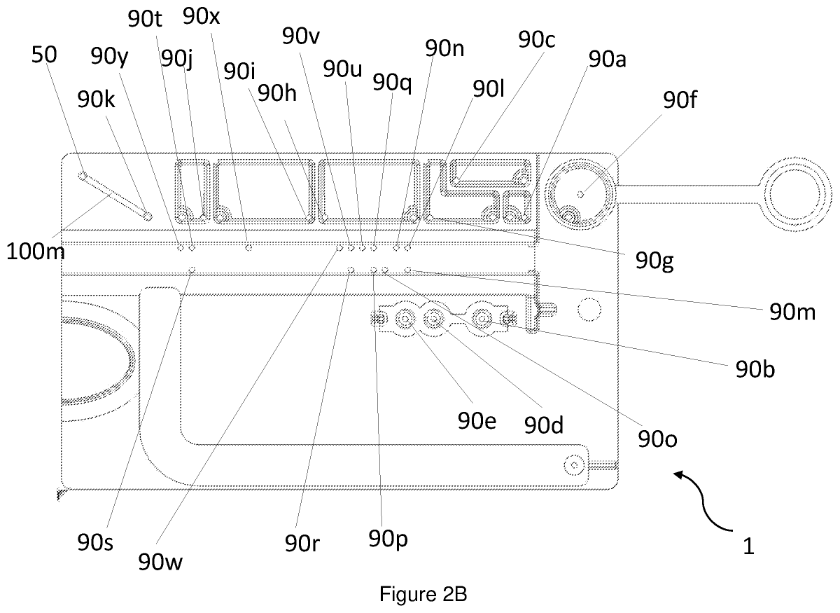

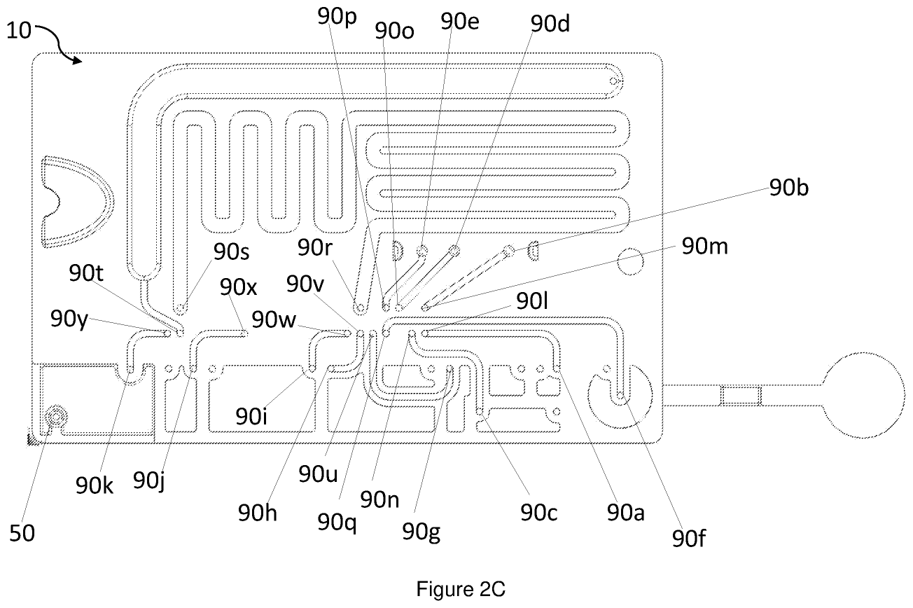

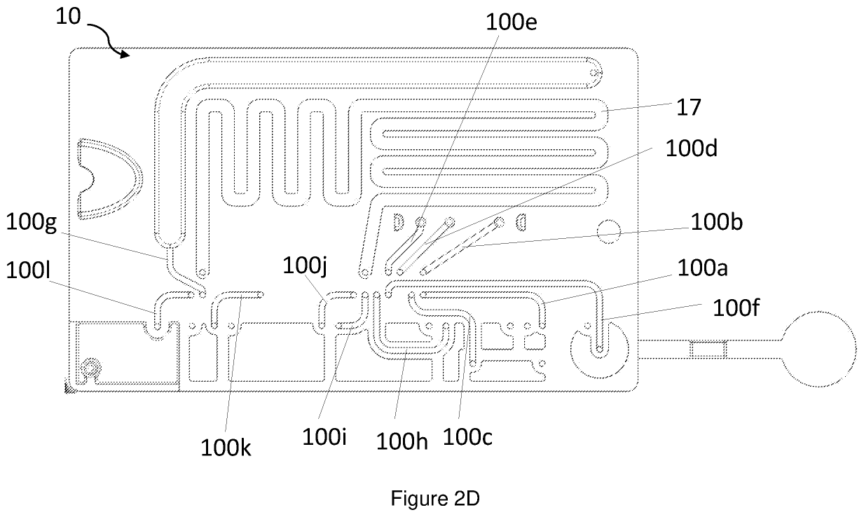

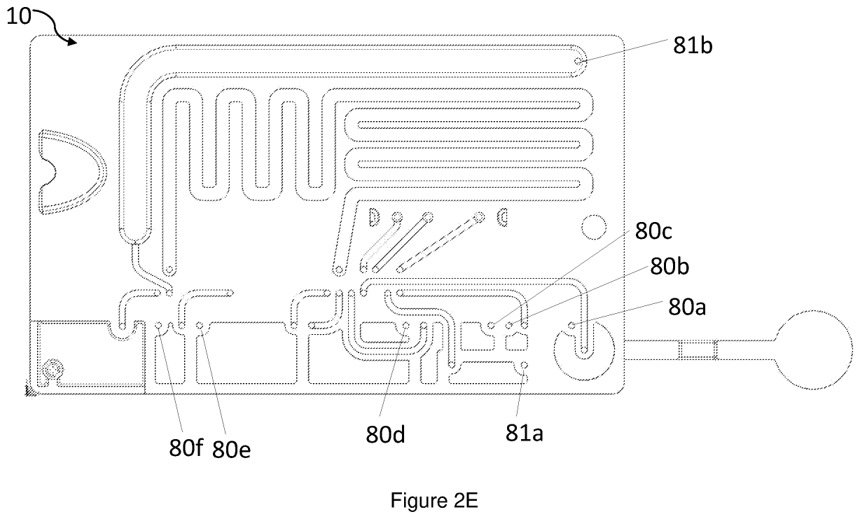

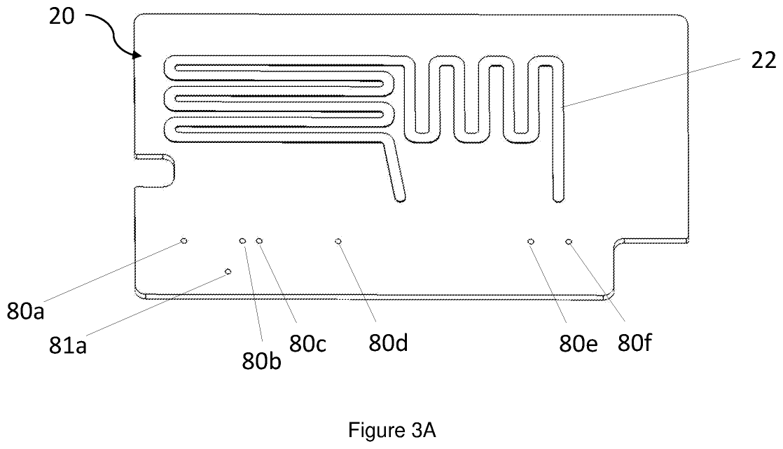

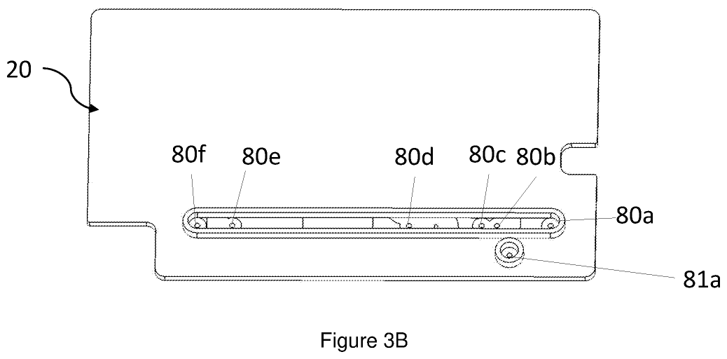

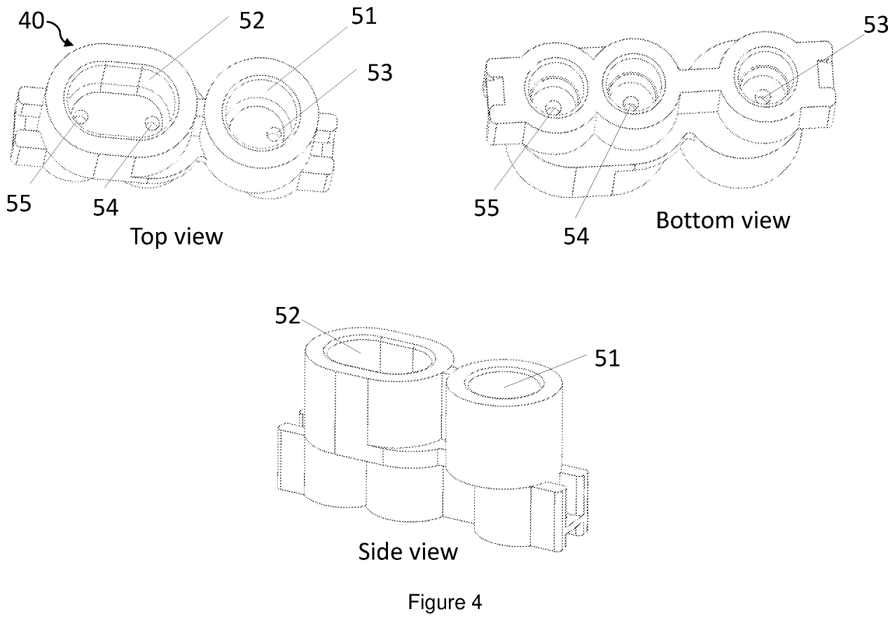

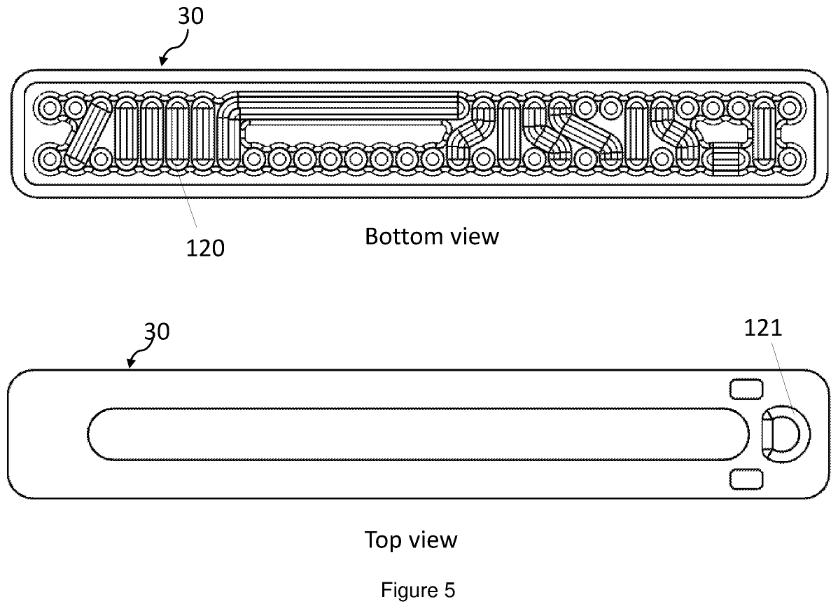

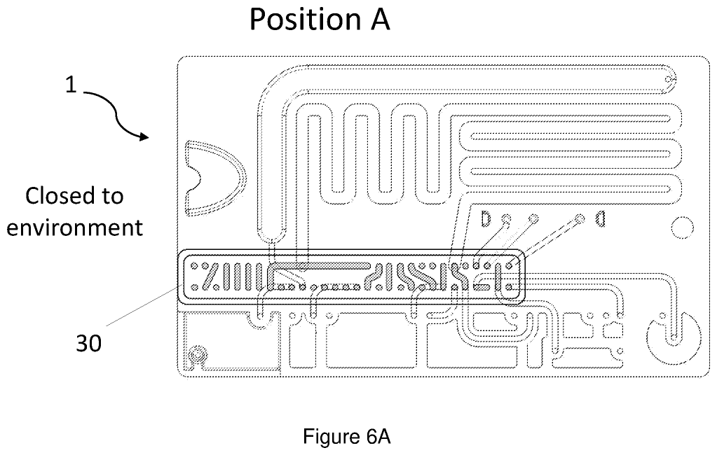

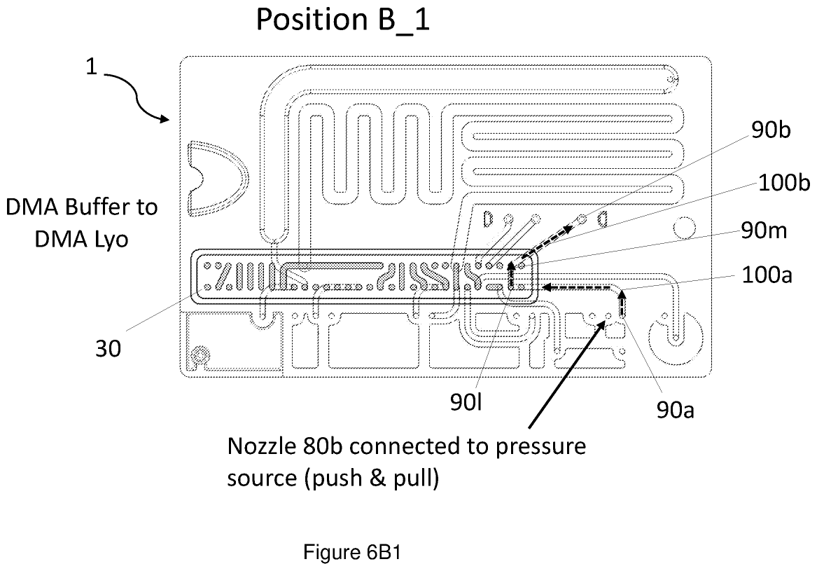

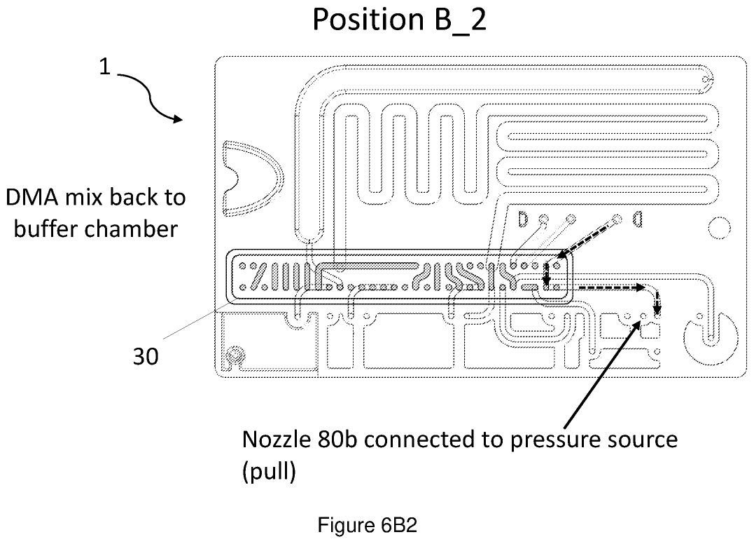

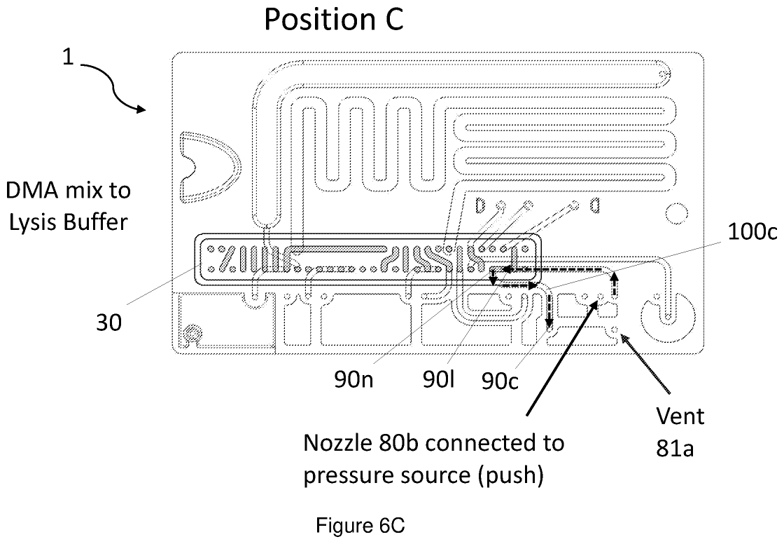

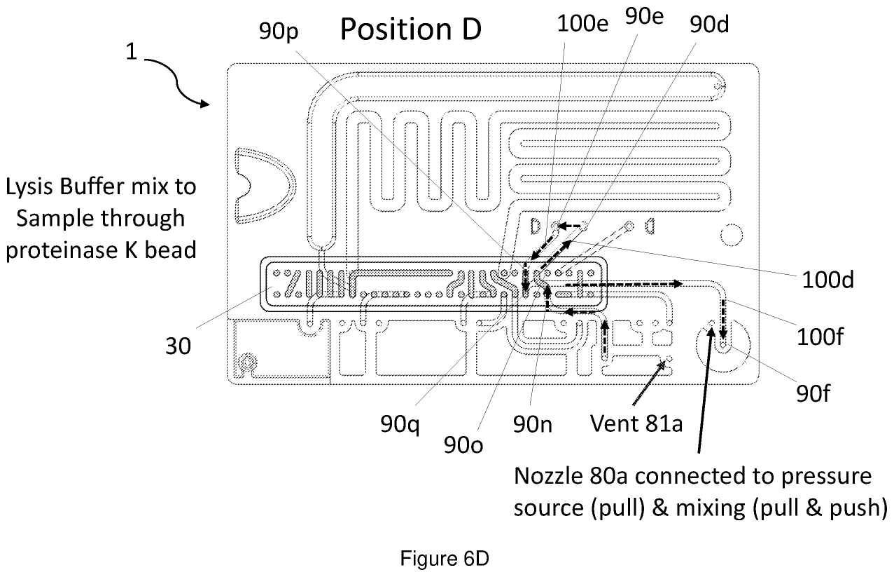

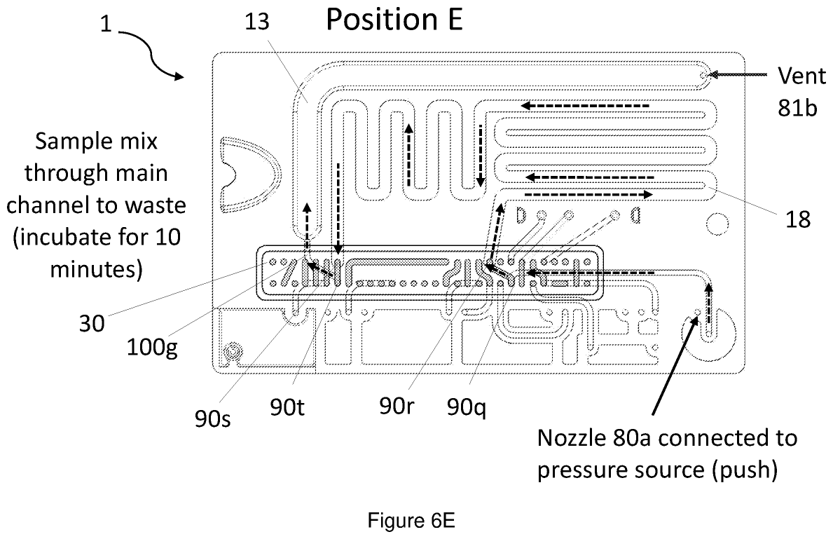

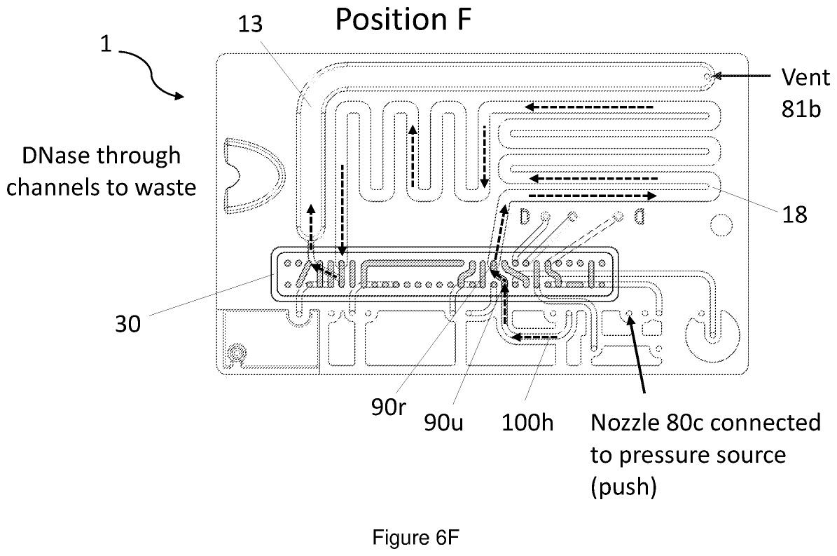

FIG. 1D shows an exploded bottom view of the device of FIG. 1A.

FIG. 2A shows a perspective view of a first housing member of the device of FIG. 1A.

FIG. 2B shows a top view of the first housing member of FIG. 2A.

FIG. 2C shows a first bottom view of the first housing member of FIG. 2A.

FIG. 2D shows a second bottom view of the first housing member of FIG. 2A.

FIG. 2E shows a third bottom view of the first housing member of FIG. 2A.

FIG. 3A shows a top view of the second housing member of the device of FIG. 1A.

FIG. 3B shows a bottom view of the second housing member of FIG. 3A.

FIG. 4 shows various views of a dry agent capsule of the device of FIG. 1A.

FIG. 5 shows top and bottom views of a slider of the device of FIG. 1A.

FIGS. 6A-6J show various positions of the slider of FIG. 5 along a track and respective fluid flows.

FIG. 7A shows a top perspective view of a device for isolating a biological material according to another example embodiment.

FIG. 7B shows a bottom perspective view of the device of FIG. 7A.

FIG. 8A shows a perspective view of a first housing member of the device of FIG. 7A.

FIG. 8B shows a bottom view of the first housing member of FIG. 8A.

FIG. 9 shows a top perspective view of the second housing member of the device of FIG. 7A.

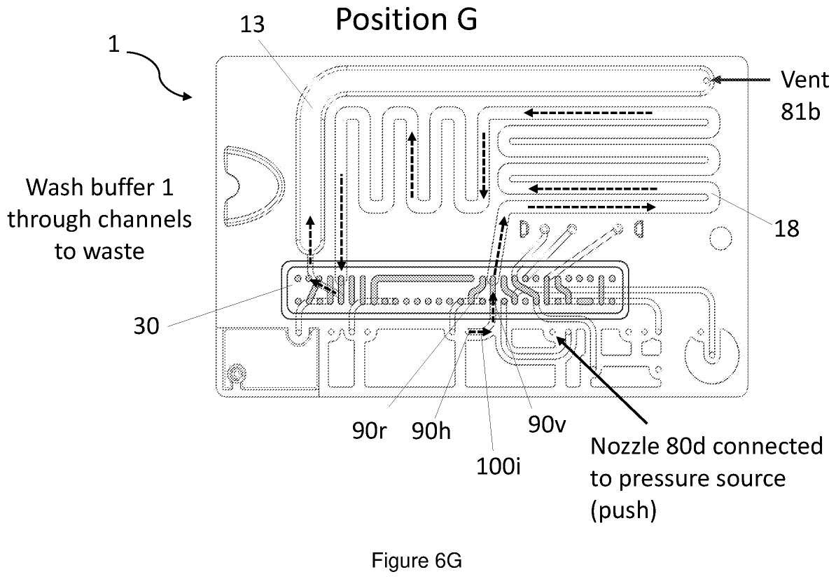

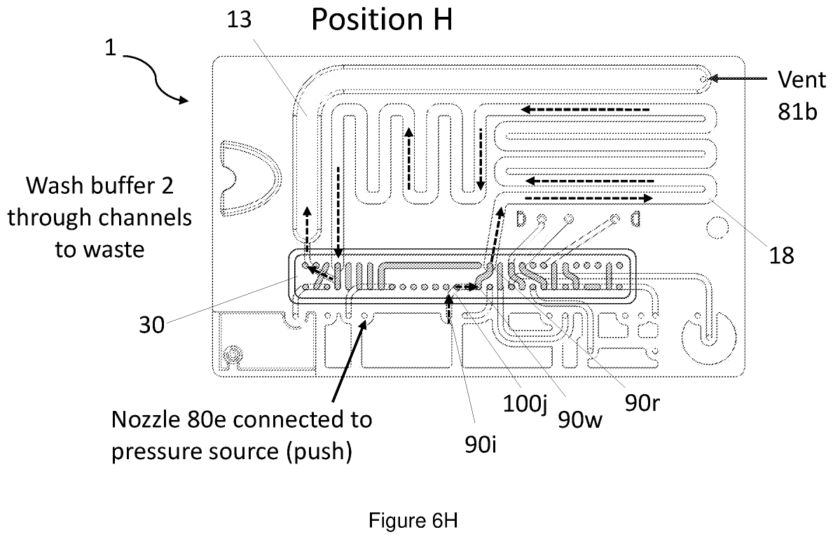

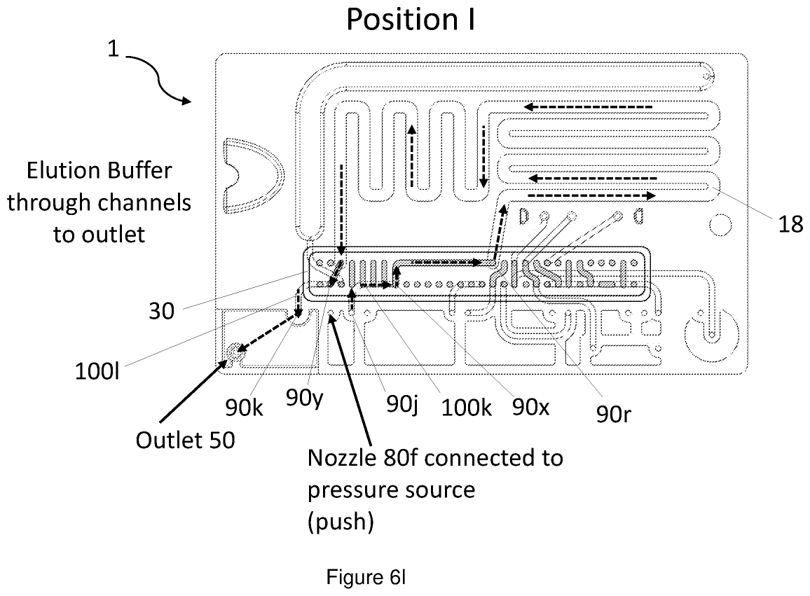

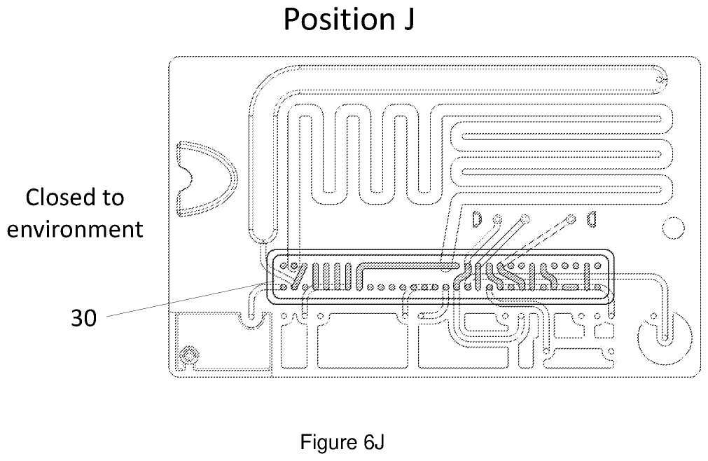

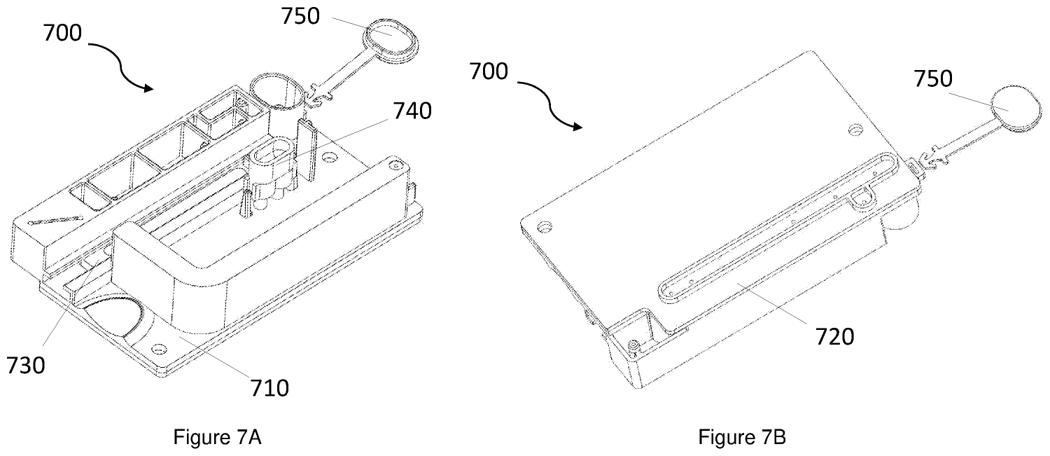

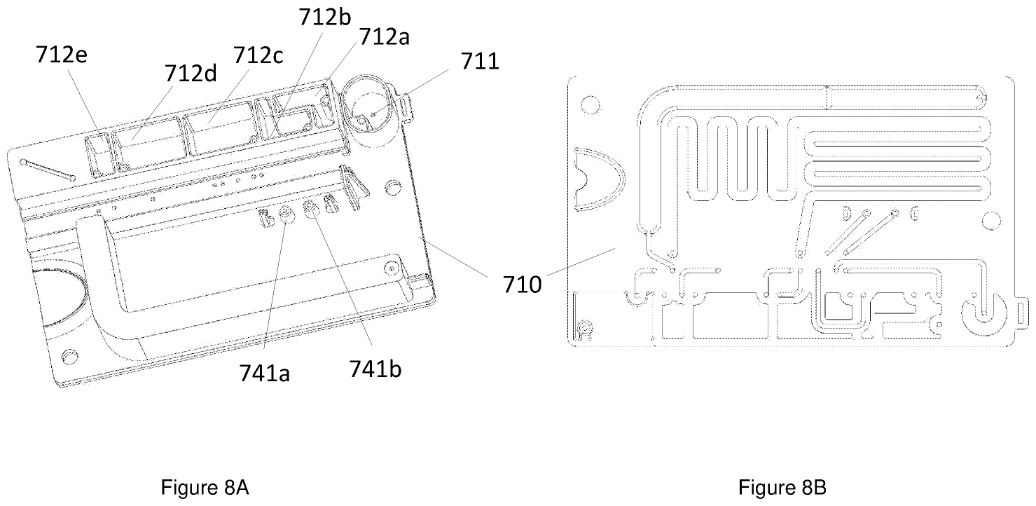

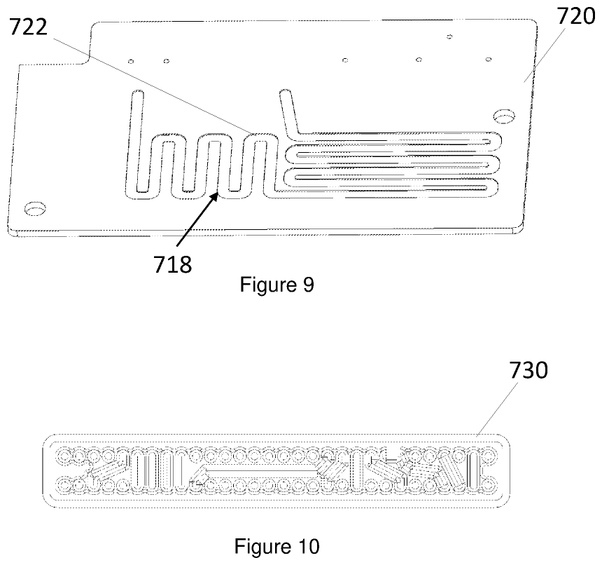

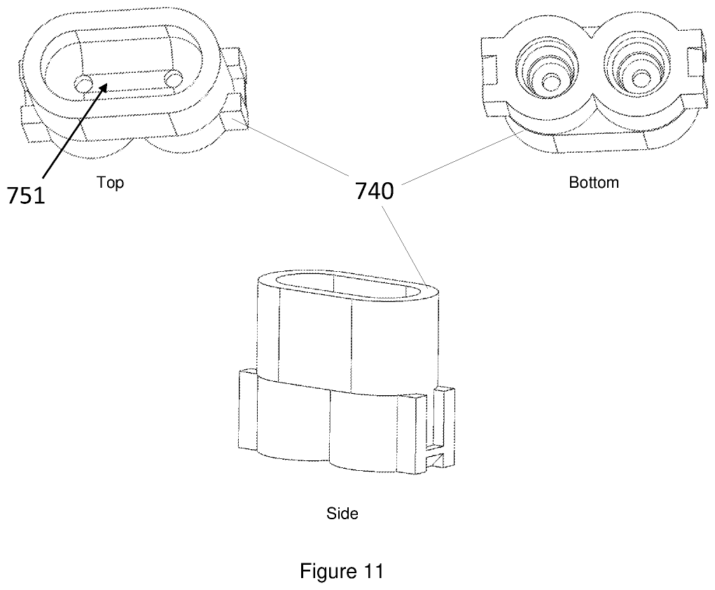

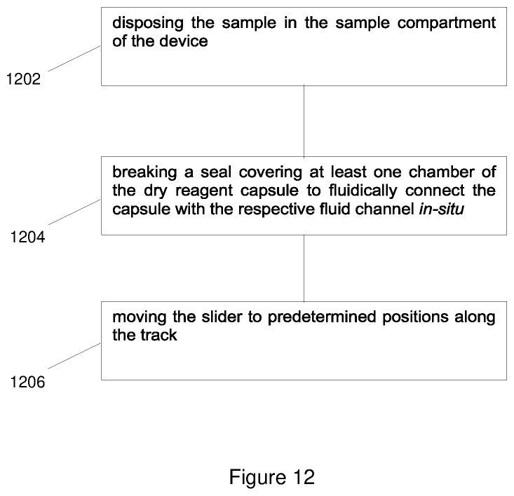

FIG. 10 shows a bottom view of a slider of the device of FIG. 7A.

FIG. 11 shows various views of a dry agent capsule of the device of FIG. 7A.

FIG. 12 shows a flow chart illustrating a method of isolating a biological material from a sample according to an example embodiment.

### DETAILED DESCRIPTION

The present disclosure provides a device for performing extraction and purification of a biological material, e.g. nucleic acids such as deoxyribonucleic acid (DNA) and ribonucleic acid (RNA), from a sample in a closed system. An example of the device is a disposable cartridge containing a sliding valve. Reagent fluids are stored onboard, along with foil sealed capsules that isolate lyophilized beads from any potential fluids/fluid vapors. When the cartridge is loaded onto an instrument, the instrument actuates the sliding valve to align unique channels with outlets from the reagent well. The valve is configured to have an “off” position at the beginning and end of travel. Air nozzles are placed in each reagent well and connected to an external pressure source/actuator to control a fluid flow. Vented air nozzles are sealed with a hydrophobic filter to prevent leaking of liquid reagents and then with a foil to dead end the chimney, preventing any fluid from saturating a protective hydrophobic vent. The instrument pierces this foil when the cartridge is inserted. A foil-sealed capsule contains lyophilized bead or dry reagent. The capsule is retained on the cartridge by press fit posts. As the instrument actuates downward, the bottom foil of the capsule is pierced by drafted posts that then seal on vertical channels. These are isolated by the sliding valve until it is moved to a position that allows fluid to flow into the capsule.

The device is configured to process the extraction and isolation of nucleic acids from samples, such as biological samples, environment samples, etc. The cartridge is configured to mix reagents with a sample for extracting nucleic acids from the sample, to bind/isolate nucleic acids in the fluidic channel and to elute nucleic acids.

In an example, the fluidic channel is coated with a chemical functional group (e.g. amino (—NH₂) group) to capture extracted nucleic acids using cross-linking agents (e.g. homobifunctional imidoesters). The device is configured to wash away cell and protein residues, any other contaminants while nucleic acids are bound to the surface and to store liquid waste in an on-board waste well. The device is configured to elute purified nucleic acids by treating the fluidic channel with an elution buffer (e.g. a buffer solution with pH >10.6) that releases nucleic acids from the fluidic channel. The eluted nucleic acid is then dispensed into an external container, such as an Eppendorf tube, to be used for the downstream process and/or analysis.

In the description that follow, the device and method are described in relation to the isolation of a nucleic acid from a sample, but it will be appreciated that the structure of the device and its working principles can be applied to the isolation of other biological materials.

FIGS. 1A-1D show various views of a device for isolating a biological material, in the form of a nucleic acid (NA) extraction cartridge 1, according to an example embodiment. The NA extraction cartridge 1 comprises a first housing member 10 (hereinafter also referred to as main body 10), a second housing member 20 (hereinafter also referred to as lid plate 20), a slider 30 (hereinafter also referred to as sliding valve 30), and a dry reagent capsule 40. These parts are typically made of plastic including, but not limited to, acrylonitrile Butadiene Styrene (ABS), polyethylene (HDPE or LDPE), polycarbonate (PC), polypropylene (PP), polyamide, Cyclic olefin copolymer (COC), thermoplastic elastomer, Polyethylene terephthalate (PET), Polyethylene terephthalate glycol (PETG), SMMA, polymethyl methacrylate (PMMA), etc.

The main body 10 includes a sample compartment 11, a reagent reservoir 12 and a waste compartment 13. A cap 14, which is configured to seal the sample compartment 11 in a fluid-tight manner, is attached to the main body 10 of the cartridge 1 via a hinge 16. Reagent compartments of the reagent reservoir 12 contain various liquid reagents for extracting and purifying NA molecules from a sample (as described in further details below) and are sealed with foil 60. The foil 60 includes a moisture-impermeable membrane, which is typically a heat-sealable moisture barrier film, such as mylar foil and metalized plastic film. An outlet 50 is configured to dispense eluted NA to a container, such as an Eppendorf tube.

As discussed below with reference to FIGS. 6A-6J, the sliding valve 30 can move laterally along the sliding valve channel or track 15 to different positions to allow fluidic connection for different stages of NA extraction/purification process.

A NA binding channel 18 is formed by a NA binding groove 17 of main body 10 and a corresponding NA binding ridge 22 of the lid plate 20. The channel is formed by bonding the lid plate 20 to the bottom side of main cartridge 10. Various bonding methods such as chemical (adhesive) bonding, solvent bonding, laser welding, ultrasonic welding can be used to bond the two parts. In alternate embodiments, the NA binding channel 18 may be formed on either the main body 10 or the lid plate 20.

Vented air inlets are covered with a respective liquid-impermeable membrane 70-72, which includes a hydrophobic filter to prevent leaking of liquid reagents, and then sealed with foil 63, 64 to prevent any fluid from saturating a protective hydrophobic vent during storage. The instrument pierces the foil 63, 64 when the cartridge 1 is inserted.

In the embodiment shown in FIGS. 1A-1D, the dry reagent capsule 40 has two columns to store lyophilized beads. Bottom and top sides of the dry reagent capsule 40 are covered with foil 61 and 62, respectively, to provide a moisture barrier to protect dry reagents during storage. Once the cartridge is inserted in the instrument, the instrument actuates downward, and the bottom foil 61 of the capsule 40 is pierced by posts that then seal on vertical channels.

FIGS. 2A-2E show various views of the first housing member or main body 10 of the NA extraction cartridge 1, and FIGS. 3A-3B show various views of the second housing member or lid plate 20 of the cartridge 1. Reagent reservoir 12 is divided into a plurality of compartments or wells 12a-12f that contain various liquid reagents which are preloaded. In an example, compartment 12a contains a hydration buffer for dry beads, compartment 12b contains a lysis buffer, compartment 12c contains DNase, compartment 12d contains wash buffer 1, compartment 12e contains wash buffer 2, and compartment 12f contains an elution buffer, respectively. In some applications, compartment 12c may be empty and not in use, but it will be appreciated that the end-user can optionally add a desired reagent.

As shown in FIGS. 2A and 2E, an air nozzle 80a is disposed in the sample compartment 11, and an air nozzle 80b-80f is placed in each reagent well of the reagent reservoir 12. Air nozzles 80a-80f span the extraction cartridge 1 from the top side through the bottom side of the main body 10 then the top side of the lid plate 20 to the bottom side of the lid plate 20 (FIGS. 3A and 3B). While not shown in the drawings, air nozzles 80a-80f are connected to an external pressure source/actuator, such as syringe pumps or a suitable pneumatic source, to control a fluid flow to or from the associated well, thereby pushing or pulling liquid from/to the sample compartment, reagent compartments and NA binding channel. Further, an air vent 81a is disposed in one of reagent wells of the reagent reservoir 12 and another air vent 81b is disposed in the waste compartment 13, respectively, to allow the fluidic movement.

As also described with reference to FIG. 1, vertical channels 41a-41c in the form of hollow posts are provided on the main body 10 for connection with dry reagent capsule 40.

When the cartridge 1 is inserted in the instrument, the instrument actuates downward, the bottom foil of the capsule 40 is pierced by these hollow posts that then seal on vertical channels 41a-41c. In other words, the dry reagent capsule 40 in the example embodiments is fluidically connected to a respective fluid channel in-situ.

With reference to FIG. 4, the dry reagent capsule 40 in this example includes chambers 51, 52 each configured to contain a respective dry reagent in the form of lyophilized beads. A different number of chambers may be used in alternate embodiments. Further, holes 53, 54, and 55 are provided at the bottom of the chambers 51, 52 for connection with the vertical channels 41a, 41b and 41c, respectively.

The placement of vertical channels is shown in FIGS. 2B and 2C. A plurality of vertical channels 90a-90y span from the top side to the bottom of the main body 10. In addition, as shown in FIG. 2D, a plurality of horizontal grooves 100a-1001 are defined in the bottom side of the main body 10, while a horizontal groove 100m (FIG. 2B) is defined in the top side of the main body 10. Horizontal fluid channels are formed by bonding the lid plate 20 to the bottom side of main body 10. Each groove 100a-100m is configured to connect a selected one of the vertical channels 90a-90k to a selected one of the vertical channels 90l-90y and NA outlet 50. For example, groove 100a connects vertical channel 90a with vertical channel 90l.

It can also be seen from FIGS. 2A-2E that the sample compartment 11 and each compartment of the reagent reservoir are configured to be fluidically connected to respective fluid channels, and each fluid channel has a respective end terminating at the groove or track 15 disposed on main body 10. For example, vertical channels 90l-90y are located underneath the sliding valve 30 when the sliding valve 30 is disposed in the track 15.

FIG. 5 shows various views of the sliding valve 30 according to an example embodiment. The sliding valve 30 has a plurality of different channels 120 which align with a plurality of vertical channels (90l-90y). The sliding valve 30 can slide from one end of the track 15 to the other by pulling by the holder 121. For example, in an automated system, an actuator can be used to drive the sliding valve 30 through precise distances such that a selected one of the connecting channels 120 can connect ends of selected ones of the fluid channels formed by the horizontal grooves 100a-1001 based on a position of the slider 30 along the track 15.

With reference to FIGS. 6A-6J, an example operation of the cartridge 1 for nucleic acid extraction and purification is now described. A sample is disposed in the sample compartment 11. In this operation, an external pneumatic/pressure source, which is connected to air nozzles 80a-80f (FIGS. 3A-3B), can be used to effect fluid flows. It will be appreciated that the device can also be used for the isolation of other biological materials, e.g. by appropriate changes to the reagents.

In FIG. 6A (position A), the slider or sliding valve 30 is in the initial or reference position, in which all channels are closed. For example, the cartridge 1 may be inserted into an instrument or system with the slider 30 being in this position. From this position, the slider 30 can be moved to other positions in a series of discrete steps.

During the subsequent operation, air pressure is equally applied to reagent wells or compartments via air nozzles 80a-80f. However, the liquid/reagent only flows along certain fluidic channels opened up by moving the sliding valve 30.

In FIGS. 6B1 and 6B2 (positions B1 and B2), the sliding valve 30 is positioned to make a channel between vertical channel 90l and vertical channel 90m such that a fluidic passage is formed between the compartment 12a and dry reagent chamber 51. A hydration buffer is pushed from compartment 12a to dry reagent chamber 51 through horizontal channel 100a (connected to vertical channel 90a and vertical channel 90l), horizontal channel 100b (connected to vertical channel 90m and vertical channel 90b) and hole 53 (FIG. 4) at the bottom of the dry reagent capsule chambers 51 to dissolve a first dry reagent in the form of lyophilized beads (in and embodiment dimethyl adipimidate (DMA) or a suitable homobifunctional imidoester crosslinker) in the chamber 51 then pull back to compartment 12a. For example, when the hydration buffer reaches the chamber 51, a combination of positive and negative pressures applied by the external source via air nozzle 80b can result in mixing of the hydration buffer with the dry reagent, before the solution is withdrawn to compartment 12a.

In FIG. 6C (position C), the sliding valve 30 is positioned to make a channel between vertical channel 90l and vertical channel 90n such that a fluidic passage is formed between compartment 12a and compartment 12b. The crosslinker solution is pushed from compartment 12a to compartment 12b, which contains a lysis buffer, through horizontal channel 100a (connected to vertical channel 90a and vertical channel 90l) and horizontal channel 100c (connected to vertical channel 90c and vertical channel 90n) to mix with the lysis buffer. The external pressure is provided via air nozzle 80b and the air vent 81a can promote fluidic movement by releasing the pressure inside compartment 12b.

In FIG. 6D (position D), the sliding valve 30 is positioned to make a channel between vertical channels 90n and 90o and between vertical channels 90p and 90q such that a fluidic passage is formed between the compartment 12b, the dry reagent chamber 52, and the sample compartment 11. The crosslinker solution+lysis buffer from compartment 12b is pushed to dry reagent chamber 52 through horizontal channel 100c (connected to vertical channel 90c and vertical channel 90n), horizontal channel 100d (connected to vertical channel 90o and vertical channel 90d) and hole 54 (FIG. 4) at the bottom of the dry reagent capsule chamber 52 to dissolve a second dry reagent in the form of lyophilized beads (in an embodiment proteinase K). Then, the mixed solution is pushed from dry reagent chamber 52 to sample compartment 11 through hole 55 (FIG. 4), horizontal channel 100e (connected to vertical channel 90e and vertical channel 90p) and horizontal channel 100f (connected to vertical channel 90f and vertical channel 90q) to mix with a sample contained in the sample compartment 11. At this position, the external pressure is applied via air nozzle 80a.

In FIG. 6E (position E), the sliding valve 30 is positioned to make a channel between vertical channels 90q and 90r and between vertical channels 90s and 90t such that a fluidic passage is formed between the sample compartment 11 and the NA binding channel 18. The mixed solution of lysis buffer+sample is pushed from sample compartment 11 to NA binding channel 18 through horizontal channel 100f (connected to vertical channel 90f and vertical channel 90q) then via vertical channel 90r. NA binding channel 18 is connected to waste compartment 13 via vertical channel 90s then through horizontal channel 100g (connected to vertical channel 90t and waste compartment 13). At this position, the external pressure is applied via air nozzle 80a. The air vent 81b promotes fluidic movement by releasing the pressure inside sample compartment 11. Then, the external pressure source is switched off, therefore no liquid is moving to the waste compartment 13 and the sample solution is incubated for a predetermined time (e.g. 10 minutes) to lyse the cells and to bind extracted NA to a surface of the NA binding channel 18. The channel 18 can be optionally heated by a heater positioned underneath the cartridge 1 and provided with the instrument into which the cartridge 1 is inserted. The incubation period may vary in alternate embodiments, e.g. depending on the material of the sample.

In FIG. 6F (position F), the sliding valve 30 is positioned to make a channel between vertical channels 90u and 90r and between vertical channels 90s and 90t such that a fluidic passage is formed between the reagent compartment 12c, NA binding channel 18 and waste compartment 13. A liquid reagent (DNase) from reagent compartment 12c is pushed to NA binding channel 18 through horizontal channel 100h (connected to vertical channel 90g and vertical channel 90u) then via vertical channel 90r to react with the surface-bound NA. The left-over sample solution (lysed sample minus NA molecules) is pushed from NA binding channel 18 to waste compartment 13 via vertical channel 90s then through horizontal channel 100g (connected to vertical channel 90t and waste well 13). At this position, the external pressure is applied via air nozzle 80c. In some embodiments, the use of the DNase treatment may be optional and can be skipped.

In FIG. 6G (position G), the sliding valve is 30 positioned to make a channel between vertical channels 90v and 90r and between vertical channels 90s and 90t such that a fluidic passage is formed between reagent compartment 12d, NA binding channel 18 and waste compartment 13. A first wash buffer is pushed from reagent compartment 12d to NA binding channel 18 through horizontal channel 100i (connected to vertical channel 90h and vertical channel 90v) then via vertical channel 90r to wash the surface-bound NA. The left-over sample solution (lysed sample minus NA molecules) from NA binding channel 18 is pushed to waste compartment 13 via vertical channel 90s then through horizontal channel 100g (connected to vertical channel 90t and waste well 13). At this position, the external pressure is applied via air nozzle 80d.

In FIG. 6H (position H), the sliding valve 30 is positioned to make a channel between vertical channels 90w and 90r and between vertical channels 90s and 90t such that a fluidic passage is formed between reagent compartment 12e, NA binding channel 18 and waste compartment 13. A second wash buffer is pushed from reagent compartment 12e to NA binding channel 18 through horizontal channel 100j (connected to vertical channel 90i and vertical channel 90w) then via vertical channel 90r to wash the surface-bound NA. The left-over sample solution (lysed sample minus NA molecules) from NA binding channel 18 is pushed to waste compartment 13 via vertical channel 90s then through horizontal channel 100g (connected to vertical channel 90t and waste well 13). At this position, the external pressure is applied via air nozzle 80e.

In FIG. 6I (position I), the sliding valve 30 is positioned to make a channel between vertical channels 90x and 90r and between vertical channels 90s and 90y such that a fluidic passage is formed between reagent compartment 12f, NA binding channel 18 and outlet 50. An elution buffer is pushed from reagent compartment 12f to NA binding channel 18 through horizontal channel 100k (connected to vertical channel 90j and vertical channel 90x) then via vertical channel 90r to elute NA from the binding channel 18. Then, the eluted NA from NA binding channel 18 is pushed to NA outlet 50 via vertical channel 90s then through horizontal channel 100l (connected to vertical channel 90k and vertical channel 90y) and horizontal channel 100m (connected vertical channel 90k and NA outlet 50). The eluted NA can be collected at the outlet 50 by suitable means. At this position, the external pressure is applied via air nozzle 80f.

In FIG. 6J (position J), which is at the end of the process, the sliding valve 30 is position to close all vertical channels. In this position, the cartridge 1 can be withdrawn from the instrument and suitably disposed.

FIGS. 7A-7B show top and bottom perspective views of a device for isolating a biological material, in the form of a nucleic acid (NA) extraction cartridge 700, according to an alternate embodiment. The NA extraction cartridge 700 is largely similar to the NA extraction cartridge described above with reference to FIGS. 1-6, and comprises a first housing member 710 (hereinafter also referred to as main body 710), a second housing member 720 (hereinafter also referred to as lid plate 720), a slider 730 (hereinafter also referred to as sliding valve 730), a dry reagent capsule 740, and a cap 750. FIG. 8A-8B shows perspective and bottom views of the first housing member 710. FIG. 9 shows a top perspective view of the second housing member 720. FIG. 10 shows a bottom view of the slider 730, and FIG. 11 shows various views of the dry agent capsule 740.

With reference to FIG. 8A, the first housing member 710 differs from the first housing member 10 described above in a number of features. Firstly, the number of reagent compartments or wells of the first housing member 710 is reduced to five. In an example, compartment 712a contains a lysis buffer, compartment 712b contains a 1^{st} wash buffer, compartment 712c contains a 2^{nd} wash buffer, compartment 712d contains 3^{rd} wash buffer, and compartment 712e contains an elution buffer, respectively. In other words, a hydration buffer chamber is not required in this embodiment. Secondly, the number of vertical channels in the form of hollow posts provided on the first housing member 710 for connection with dry reagent capsule 740 is reduced from three to two (see 741a and 741b in FIG. 8A), since the number of dry reagent chambers of the dry reagent capsule 740 is one (see 751 in FIG. 10). For example, the chamber 751 can contain the at least one dry reagent (e.g. both first and second dry reagents) in the form of lyophilized beads. Accordingly, the number and positions of the horizontal channels of the first housing member 710 and connecting channels of the slider 730 are adapted to accommodate the above changes.

Other changes in the NA extraction cartridge 700 compared to the NA extraction cartridge 10 include forming the NA binding channel 718 by groove 722 in the second housing member 720 (see FIG. 9) instead of a ridge.

Operation of the NA extraction cartridge 700 is similar to that of the NA extraction cartridge 10, except that the steps as described above with reference to FIGS. 6B1-6B2, 6C and 6D are combined into a single step. In this step, the lysis buffer from compartment 712a is pushed through respective fluid channels and connecting channel therebetween to the dry reagent chamber 751 where the lysis buffer can dissolve the one or multiple dry reagents present in the chamber. For example, when the lysis buffer reaches the chamber 751, a combination of positive and negative pressures applied by the external source via an air nozzle can result in mixing of the lysis buffer with the dry reagents, before the solution is withdrawn to the sample compartment 711 to mix with the sample contained in the sample compartment 711. One of the vertical channel 741a, 741b is used for injecting the lysis buffer into the chamber 751 while the other is used for withdrawing the mixed solution from the chamber 751. The lysis buffer in this embodiment can be, for example, a mixture of the lysis buffer and hydration buffer of the embodiment in FIGS. 1-6, or a new recipe capable of dissolving lyophilized beads and lysing the biomolecules in the sample. In other words, this embodiment can simplify the preparation of the reagent solution by selecting the appropriate combination of the lysis buffer and dry reagents, resulting in a reduction of two steps.

Thereafter, the NA extraction cartridge 700 can be operated in the same way as the NA extraction cartridge 10. In this embodiment, the DNase treatment step (as discussed above with reference to FIG. 6F) can be skipped, and replaced with an additional washing step as desired, i.e. 3 washing steps using the 1^{st}, 2^{nd} and 3^{rd} wash buffers respectively. The composition of the wash buffers can differ depending on the target molecules and the bodily fluids present in the sample. The wash buffers are interchangeable between this embodiment and the first embodiment of FIGS. 1-6, especially when the sample compartment for DNase treatment step in the first embodiment is used to contain a wash buffer instead. For the sake of brevity, the washing and elution steps are not repeated here.

FIG. 12 shows a flow chart 1200 illustrating a method of isolating a biological material from a sample according to an example embodiment. At step 1202, the sample is disposed in the sample compartment of the device as described above. At step 1204, a seal covering at least one chamber of the dry reagent capsule is broken to fluidically connect the capsule with the respective fluid channel in-situ. At step 1206, the slider is moved to predetermined positions along the track to successively mix at least one liquid reagents and the at least one dry reagent with the sample for extracting the biological material from the sample, bind the extracted biological material to a surface of a binding channel disposed in the device, purify the biological material bound to the surface of the binding channel, and elute the purified biological material.

As described, the isolation of nucleic acid can be performed using a compact and self-containing device which is in the form of a cartridge that can be inserted into an instrument before the process and removed from the instrument once the process is completed. In other words, the relevant liquid and dry reagents are already present in the device and external liquid handling is not necessary. Cross-contamination and maintenance can be significantly reduced. Furthermore, the use of a single slider with integrated connecting channels, together with linear movements of the slider, to selectively connect fluid channels of the device can reduce the number of moving parts and enable automated operation.

It will be appreciated by a person skilled in the art that numerous variations and/or modifications may be made to the present disclosure as shown in the specific embodiments without departing from the scope of the disclosure as broadly described. For example, suitable adjustments can be made to the reagents or sequence of operations to adapt the device for isolation of a different type of biological material. The present embodiments are, therefore, to be considered in all respects to be illustrative and not restrictive.

1. A device for isolating a biological material from a sample, the device comprising:
a housing defining a plurality of compartments and a plurality of fluid channels, wherein each compartment is configured to be fluidically connected to a respective fluid channel, and wherein each fluid channel comprises a respective end terminating at a track disposed on the housing;
a slider movable along the track, the slider comprising a plurality of connecting channels extending therethrough, wherein a selected one of the connecting channels is configured to connect ends of selected ones of the fluid channels based on a position of the slider along the track; and
a dry reagent capsule configured to be mounted to the housing, the dry reagent capsule comprising at least one dry reagent for mixing with the sample, wherein the dry reagent capsule is further configured to be fluidically connected to a respective fluid channel in-situ.

2. (canceled)

2. The device as claimed in claim 1, wherein the plurality of compartments comprises a sample compartment configured to receive the sample, a plurality of liquid reagent compartments and a waste compartment, and wherein the plurality of liquid reagent compartments are preloaded with respective liquid reagents.

3. The device as claimed in claim 2, wherein each of the sample and liquid reagent compartments comprises a respective inlet configured to be connected to pneumatic source for controlling a fluid flow to or from said compartment, the device further comprising a first pneumatic vent disposed in one of the liquid reagent compartments and a second pneumatic vent disposed in the waste compartment.

5. (canceled)

6. (canceled)

4. The device as claimed in claim 2, wherein, in a first position, the slider is configured to connect a first liquid reagent compartment containing a hydration buffer with a first chamber of the dry reagent capsule, the first chamber containing a first dry reagent, for mixing the hydration buffer with the first dry reagent to form a first solution.

5. The device as claimed in claim 4, wherein, in a second position, the slider is configured to connect the first liquid reagent compartment with a second liquid reagent compartment containing a lysis buffer, for mixing the first solution with the lysis buffer to form a second solution.

6. The device as claimed in claim 5, wherein, in a third position, the slider is configured to:
connect the second liquid reagent compartment with a second chamber of the dry reagent capsule, the second chamber containing a second dry reagent, for mixing the second solution with the second dry reagent to form a third solution; and
connect the second chamber of the dry reagent capsule with the sample compartment for mixing the third solution with the sample to form a fourth solution.

7. The device as claimed in claim 6, wherein the fluid channels comprise a binding channel, and wherein, in a fourth position, the slider is configured to connect the sample compartment with the binding channel to store the fourth solution in the binding channel for a predetermined period for extracting the biological material from the fourth solution and binding the extracted biological material to a surface of the binding channel.

8. The device as claimed in claim 7, wherein, in a fifth position, the slider is configured to:
connect a third liquid reagent compartment containing a first wash buffer with the binding channel to wash the biological material bound to the surface of the binding channel; and
connect the binding channel with the waste compartment for discarding a first waste solution without the biological material to the waste compartment.

9. The device as claimed in claim 8, wherein, in a sixth position, the slider is configured to:
connect a fourth liquid reagent compartment containing a second wash buffer with the binding channel to wash the biological material bound to the surface of the binding channel; and
connect the binding channel with the waste compartment for discarding a second waste solution without the biological material to the waste compartment.

10. The device as claimed in claim 9, wherein, in a seventh position, the slider is configured to:
connect a fifth liquid reagent compartment containing an elution buffer with the binding channel to elute the biological material from the surface of the binding channel; and
connect the binding channel with an outlet for collecting the eluted biological material.

11. The device as claimed in claim 2, wherein, in a first position, the slider is configured to:
connect a first liquid reagent compartment containing a lysis buffer with a chamber of the dry reagent capsule, the chamber containing the at least one dry reagent, for mixing the lysis buffer with the at least one dry reagent to form a reagent solution; and
connect the chamber of the dry reagent capsule with the sample compartment for mixing the reagent solution with the sample to form a sample solution.

12. The device as claimed in claim 11, wherein the fluid channels comprise a binding channel, and wherein, in a second position, the slider is configured to connect the sample compartment with the binding channel to store the sample solution in the binding channel for a predetermined period for extracting the biological material from the sample solution and binding the extracted biological material to a surface of the binding channel.

13. The device as claimed in claim 12, wherein, in a third position, the slider is configured to:
connect a second liquid reagent compartment containing a first wash buffer with the binding channel to wash the biological material bound to the surface of the binding channel; and
connect the binding channel with the waste compartment for discarding a first waste solution without the biological material to the waste compartment.

14. The device as claimed in claim 13, wherein, in a fourth position, the slider is configured to:
connect a third liquid reagent compartment containing a second wash buffer with the binding channel to wash the biological material bound to the surface of the binding channel; and
connect the binding channel with the waste compartment for discarding a second waste solution without the biological material to the waste compartment.

15. The device as claimed in claim 14, wherein, in a fifth position, the slider is configured to:
connect a fourth liquid reagent compartment containing a third wash buffer with the binding channel to wash the biological material bound to the surface of the binding channel; and
connect the binding channel with the waste compartment for discarding a third waste solution without the biological material to the waste compartment.

16. The device as claimed in claim 15, wherein, in a sixth position, the slider is configured to:
connect a fifth liquid reagent compartment containing an elution buffer with the binding channel to elute the biological material from the surface of the binding channel; and
connect the binding channel with an outlet for collecting the eluted biological material.

17. The device as claimed in claim 7, wherein the at least one dry reagent comprises lyophilized beads containing a crosslinker selected to attach to the biological material, and wherein the surface of the binding channel is coated with a functional group selected to attach to the crosslinker.

21. (canceled)

18. An automated biological material extraction system comprising:
a receptacle configured to receive the device as claimed in claim 2;
a pressure source configured to control a fluid flow to and from a selected compartment of the sample compartment and liquid reagent compartments;
an actuator configured to move the slider of the device to predetermined positions along the track; and
a mechanism configured to exert a force on the dry reagent capsule to break a seal covering at least one chamber of the capsule to fluidically connect the capsule with the respective fluid channel in-situ.

23. (canceled)

24. (canceled)

19. A method of isolating a biological material from a sample, the method comprising:
disposing the sample in the sample compartment of the device as claimed in claim 2;
breaking a seal covering at least one chamber of the dry reagent capsule to fluidically connect the capsule with the respective fluid channel in-situ; and
moving the slider to predetermined positions along the track to:
mix the liquid reagents and the at least one dry reagent with the sample for extracting the biological material from the sample;
bind the extracted biological material to a surface of a binding channel disposed in the device;
purify the biological material bound to the surface of the binding channel; and
elute the purified biological material.

26. (canceled)

20. The device as claimed in claim 12, wherein the at least one dry reagent comprises lyophilized beads containing a crosslinker selected to attach to the biological material, and wherein the surface of the binding channel is coated with a functional group selected to attach to the crosslinker.
